(19) United States
(12) Patent Application Publication
(10) Pub. No.: US 2024/0139444 A1
(43) Pub. Date: May 2, 2024

(54) VENTILATOR AND AIRBAG FOR A VENTILATOR

(71) Applicant: Simon Fraser University, Burnaby (CA)

(72) Inventors: Woo Soo KIM, Vancouver (CA); Tae-Ho KIM, Surrey (CA)

(21) Appl. No.: 18/549,751

(22) PCT Filed: Mar. 9, 2022
(86) PCT No.: PCT/CA2022/050346

§ 371 Date: Sep. 8, 2023

(30) Foreign Application Priority Data
Mar. 10, 2021 (CA) 3111724

(51) Int. Cl. A61M 16/00

(52) U.S. Cl. CPC A61M 16/0075; A61M 16/022; A61M 2205/106; A61M 2205/3334; A61M 2207/10

(57) ABSTRACT

There is described a ventilator for providing breathable air to a patient. The ventilator includes an airbag defining a longitudinal axis, having a first end, a second end, and a foldable wall extending from the first end to the second end, and being movable from a decompressed state to a compressed state by moving the first end along the longitudinal axis relative to the second end. The wall has fold lines formed therein such that, during movement of the airbag from the decompressed state to the compressed state, the wall is folded along the fold lines. The fold lines define at least one polygonal surface portion of the wall. The ventilator further includes an actuator for driving compression and decompression of the airbag, and a controller for controlling the actuator.

### FIELD OF THE DISCLOSURE

The present disclosure relates to a ventilator that may be used, for example, to assist patients suffering from breathing difficulties.

### BACKGROUND TO THE DISCLOSURE

Ventilators are life-saving medical devices used to assist in providing patients with breathable air. Respiratory pandemics such as the ongoing COVID-19 pandemic have highlighted the need for easy access to reliable ventilators. However, existing ventilators suffer from a number of drawbacks.

For example, current ventilators are often too bulky and are therefore not suitable for the transitioning of patients, for providing urgent breathing assistance in an operating theatre, or, for example, for assisting patients who wish to move about with a wheelchair. Furthermore, conventional ventilators may cost more than USD $10,000, rendering access to such ventilators more difficult in remote areas or developing parts of the world. Partly because of their high cost, traditional ventilators are typically reserved for hospital intensive care units (ICUs) and are rarely deployed outside of such settings. Further still, the airbags of existing ventilators can prematurely break down from extended usage as result of the repeated compression cycles applied to the airbag.

There is therefore an ongoing need for improved ventilators and airbags for ventilators.

### SUMMARY OF THE DISCLOSURE

According to a first aspect of the disclosure, there is described a ventilator for providing breathable air to a patient, comprising: an airbag defining a longitudinal axis, comprising a first end, a second end, and a foldable wall extending from the first end to the second end, and being movable from a decompressed state to a compressed state by moving the first end along the longitudinal axis relative to the second end, wherein the wall comprises fold lines formed therein such that, during movement of the airbag from the decompressed state to the compressed state, the wall is folded along the fold lines, and wherein the fold lines define at least one polygonal surface portion of the wall; an actuator for driving compression and decompression of the airbag; and a controller for controlling the actuator.

The ventilator may further comprise a conduit connected to the airbag for delivering the breathable air to the patient.

The at least one polygonal surface portion may comprise interconnected polygonal surface portions of the wall, and the interconnected polygonal surface portions may comprise an outer surface of the wall.

The at least one polygonal surface portion may comprise at least one planar polygonal surface portion.

The fold lines may define a Kresling pattern on an outer surface of the wall.

The at least one polygonal surface portion may comprise at least one triangular surface portion.

A first angle of the at least one triangular surface portion may be from about 30 degrees to about 34 degrees, and a second angle of the at least one triangular surface portion may be from about 38 degrees to about 45 degrees.

The first angle may be 30° and the second angle may be 42.5°.

The at least one triangular surface portion may not comprise a right angle.

The at least one triangular surface portion may comprise an obtuse angle.

The at least one triangular surface portion may comprise a right angle.

The fold lines may comprise inwardly-folding fold lines and outwardly-folding fold lines defining interconnected foldable portions of the wall. Each inwardly folding fold line may be closer to the longitudinal axis than each outwardly folding fold line.

Each foldable portion may be defined by four outwardly-folding fold lines and one inwardly folding fold line.

The four outwardly folding fold lines may define a parallelogram, and the inwardly folding fold line may extend from a first corner of the parallelogram to an opposite, second corner of the parallelogram.

The inwardly folding fold line may be perpendicular to the longitudinal axis.

The fold lines may define a Yoshimura pattern on an outer surface of the wall.

Each foldable portion may be defined by multiple inwardly-folding fold lines meeting at a point.

Each foldable portion may be defined by six outwardly-folding fold lines, and the multiple inwardly-folding fold lines may meeting at the point may consist of three inwardly-folding fold lines.

The fold lines may define a Tachi-Miura pattern on an outer surface of the wall.

The wall may be formed by three-dimensional printing.

The wall may comprise a thermoplastic.

The fold lines may be arranged such that, during movement of the airbag from the decompressed state to the compressed state, the first end rotates about the longitudinal axis relative to the second end.

The fold lines may be arranged such that, during movement of the airbag from the decompressed state to the compressed state, the first end does not rotate about the longitudinal axis relative to the second end.

The ventilator may weigh from about 3 kilograms to about 6 kilograms.

The controller may be configured to: determine a rate of flow of breathable air being delivered by the airbag; and determine whether to adjust the rate of flow of breathable air being delivered by the airbag based on a difference between the rate of flow of breathable air being delivered by the airbag and a threshold rate of flow of breathable air.

The controller may be configured to: determine a volume of breathable air being delivered by the airbag per compression cycle; and determine whether to adjust the volume of breathable air being delivered by the airbag per compression cycle based on a difference between the volume of breathable air being delivered by the airbag per compression cycle and a threshold volume of breathable air.

According to a further aspect of the disclosure, there is provided an airbag for a ventilator, comprising: a first end; a second end; and a foldable wall extending from the first end to the second end, wherein the airbag defines a longitudinal axis and is movable from a decompressed state to a compressed state by moving the first end along the longitudinal axis relative to the second end, wherein the wall comprises fold lines formed therein such that, during movement of the airbag from the decompressed state to the compressed state, the wall is folded along the fold lines, and wherein the fold lines define at least one polygonal surface portion of the wall.

According to a further aspect of the disclosure, there is provided a method of making an airbag for a ventilator, the airbag defining a longitudinal axis, comprising a first end, a second end, and a foldable wall extending from the first end to the second end, and being movable from a decompressed state to a compressed state by moving the first end along the longitudinal axis relative to the second end, wherein the wall comprises fold lines formed therein such that, during movement of the airbag from the decompressed state to the compressed state, the wall is folded along the fold lines, and wherein the fold lines define at least one polygonal surface portion of the wall.

The method may comprise printing the airbag using a three-dimensional printer.

This summary does not necessarily describe the entire scope of all aspects. Other aspects, features and advantages will be apparent to those of ordinary skill in the art upon review of the following description of specific embodiments.

### BRIEF DESCRIPTION OF THE DRAWINGS

Embodiments of the disclosure will now be described in detail in conjunction with the accompanying drawings of which:

FIG. 1 shows a ventilator according to an embodiment of the disclosure;

FIG. 2 shows an airbag in decompressed and compressed states, according to an embodiment of the disclosure;

FIG. 3 shows a fold line pattern of the airbag of FIG. 2, according to an embodiment of the disclosure;

FIG. 4 is a plot of stress as a function of strain for an airbag according to an embodiment of the disclosure;

FIG. 5 shows stress experienced by an airbag at different stages in a compression cycle, according to an embodiment of the disclosure;

FIG. 6 shows different airbags with different values for angles α and β, according to embodiments of the disclosure;

FIG. 7A is a plot of normalized volume as a function of angles α and β, according to embodiments of the disclosure;

FIG. 7B is a plot of elastic modulus as a function of peak number and angles α and β, according to embodiments of the disclosure;

FIG. 7C is a plot of compressive stress as a function of cycle number and angles α and β, according to embodiments of the disclosure;

FIGS. 8A and 8B are plots of stress as a function of strain for different airbags with different values for angles α and β, according to embodiments of the disclosure;

FIGS. 9A and 9B are plots of flow rate as a function of time for different airbags with different values for angles α and β, according to embodiments of the disclosure;

FIG. 10A is a plot of air flow as a function of cycle number, according to an embodiment of the disclosure;

FIG. 10B is a plot of flow rate as a function of time, according to an embodiment of the disclosure;

FIG. 10C is a plot of air flow as a function of time, according to an embodiment of the disclosure;

FIG. 11A shows a fold line pattern of an airbag according to an embodiment of the disclosure;

FIG. 11B shows, in a decompressed state, the airbag corresponding to the fold line pattern of FIG. 11A, according to an embodiment of the disclosure;

FIG. 12A shows an airbag transitioning from a decompressed state to a compressed state, according to an embodiment of the disclosure; and

FIG. 12B shows a fold line pattern of the airbag of FIG. 12A, according to an embodiment of the disclosure.

### DETAILED DESCRIPTION

The present disclosure seeks to provide an improved ventilator and an improved airbag for a ventilator. While various embodiments of the disclosure are described below, the disclosure is not limited to these embodiments, and variations of these embodiments may well fall within the scope of the disclosure which is to be limited only by the appended claims.

Generally, embodiments of the disclosure are directed to a ventilator that uses an airbag with a pattern of linear creases or fold lines formed thereon. The fold line pattern may enable the airbag to more efficiently fold and unfold during a compression cycle while helping to minimize the overall stress that is applied to the airbag. The fold line pattern may comprise any one or more of multiple different types of origami-based or non-origami-based patterns, such as a Kresling pattern, a Yoshimura pattern, or a Tachi-Miura pattern. The airbag may be cost-effectively produced through three-dimensional (3D) printing, and may be of a sufficiently small size that, when incorporated with other components of the ventilator, may result in a ventilator that is easily portable (and which for example may weigh between 3 and 6 kilograms). Because of the cost-effective way in which the ventilator, and in particular the airbag, may be produced, airbags having different parameters (such as different volumes, or different elastic moduli) may be rapidly and easily produced. This may enable a suitable airbag to be more rapidly deployed, depending on the needs of the particular patient.

Ventilators according to embodiments of the disclosure may be used as portable emergency devices for patients suffering, for example, from lung-related diseases such as COVID-19. Ventilators according to embodiments of the disclosure may also be particularly useful in non-ICU-type settings. For example, ventilators as described herein may be used in emergency situations by trained healthcare professionals such as military medics or paramedics for providing rapid, on-site first aid.

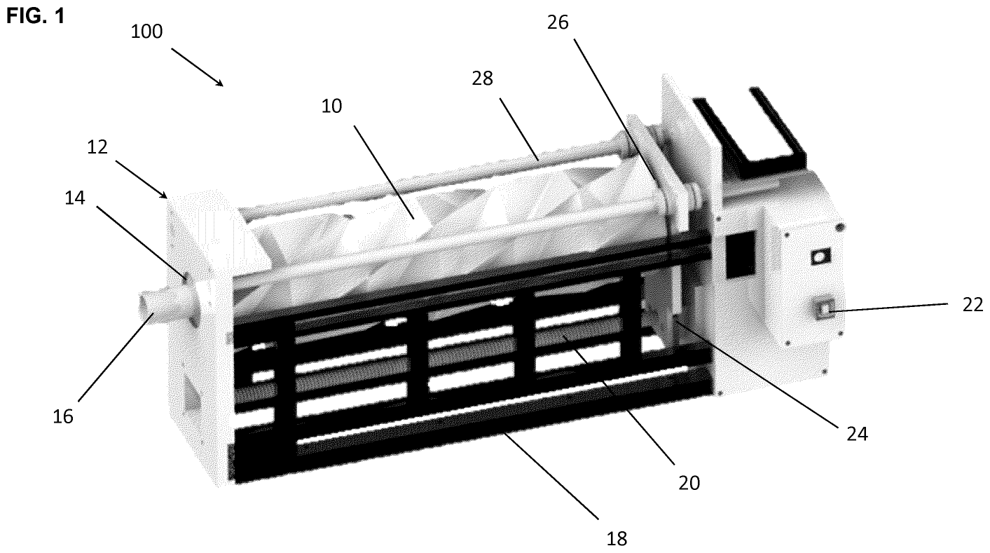

Turning now to FIG. 1, there is a shown a ventilator 100 according to an embodiment of the disclosure. Ventilator 100 comprises a housing 12 to which is connected an airbag 10 having a first end 14 and an opposite second end 26. Ventilator 100 further comprises a linear actuator 24 for driving compression and decompression of airbag 10. In particular, a motor (not shown), such as a stepper motor, may drive rotation of a lead screw 20 which in turn drives compression and decompression of airbag 10. A controller, such as a microprocessor (not shown), may be connected to linear actuator 24 via a connection port 22 and may be used to control the operation of linear actuator 24. Adjacent second end 14 of airbag 10 is provided a port 16 for connecting a breathing conduit (not shown) thereto. The breathing conduit may be connected to a patient to provide breathable air to the patient.

Housing 12 includes a pair of guide rods 28 for guiding movement of airbag 10 during compression and decompression cycles. Portions of housing 12, such as a frame 18 used to support linear actuator 24, may be 3D-printed using, for example, a fused filament fabrication (FFF) method. Ventilator 100 further comprises a display (not shown) for displaying information (such as a rate of airflow and a volume of airflow per cycle) to a user, and a flow sensor (not shown) for monitoring, for example, the rate of airflow and the volume of airflow per cycle during operation of ventilator 100.

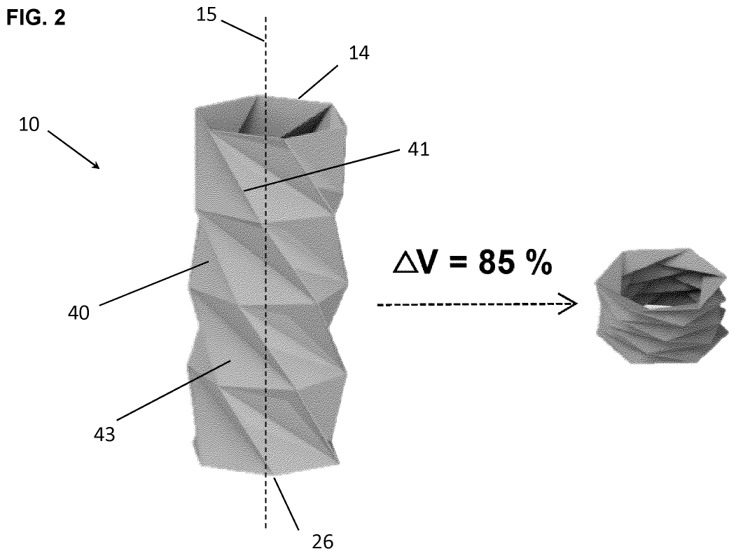

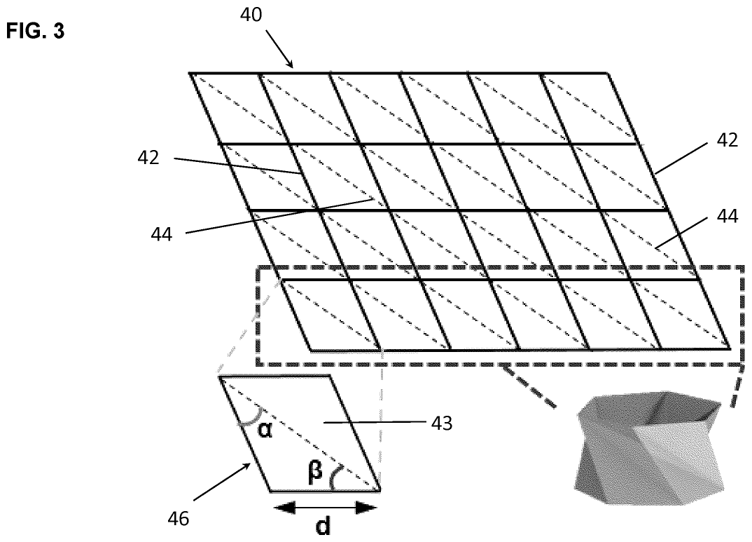

Airbag 10 will now be described in further detail in connection with FIGS. 2 and 3.

Extending between first end 14 and second end 26 of airbag 10 is a cylindrical, foldable wall 40 defining a longitudinal axis 15 of airbag 10. Wall 40 has formed in an outer surface thereof a pattern of interconnected linear creases or fold lines 41, defining a number of interconnected polygonal surface portions 43 on the outer surface of wall 40. During compression of airbag 10 (that is, when first end 14 is linearly translated toward second end 26 by virtue of linear actuator 24), wall 40 is folded along fold lines 41. FIG. 2 shows an example of airbag 10 is an uncompressed state (left) and a compressed state (right). During one compression cycle of airbag 10, a volume defined by the interior of airbag 10 may be reduced by as much as 85%. In addition, during compression of airbag 10, first end 14 is rotated relative to second end 26 about longitudinal axis 15.

The pattern of fold lines 41 on the outer surface of wall 40 may comprise a Kresling pattern, shown in more detail in FIG. 3. FIG. 3 shows in particular the three-dimensional wall 40 of FIG. 2 transposed to a two-dimensional surface. Two-dimensional wall 40 is shaped as a parallelogram comprising a grid of horizontally and generally vertically aligned folding portions 46. Each folding portion 46 is parallelogram-shaped and is defined by four outwardly-folding fold lines 42 and one inwardly-folding fold line 44 extending from a first corner of the parallelogram defined by folding portion 46 to a second, opposite corner of the parallelogram defined by folding portion 46. Generally, an outwardly-folding fold line 42 may be defined as a fold line that folds outwardly during compression of airbag 10, whereas an inwardly-folding fold line 44 may be defined as a fold line that folds inwardly during compression of airbag 10. Outwardly folding fold lines 42 are located further from longitudinal axis 15 than inwardly folding fold lines 44.

Each foldable portion 46 defined on two-dimensional wall 40 comprises a pair of planar, polygonal (in this case, triangular) surface portions 43 defined by the intersection of the inwardly folding fold line 44 with the four outwardly folding fold lines 42. The totality of interconnected, triangular surface portions 43 form the outer surface of wall 40. Each triangular surface portion 43 is defined by two angles, α and β. As described in further detail below, α and β may be adjusted to alter one or more parameters of airbag 10.

According to the embodiment of FIG. 3, a height of two-dimensional wall 40 is 200 mm and a width of two-dimensional wall 40 is 210 mm, with α=30° and β=42.5°. Airbag 10 formed by two-dimensional wall 40 may provide about 600 mL of air per compression cycle, which may be an ideal amount for an average adult male patient. According to other embodiments of the disclosure, the height and width of the wall forming the airbag may be adjusted to alter the volume of air deliverable to the patient per compression cycle.

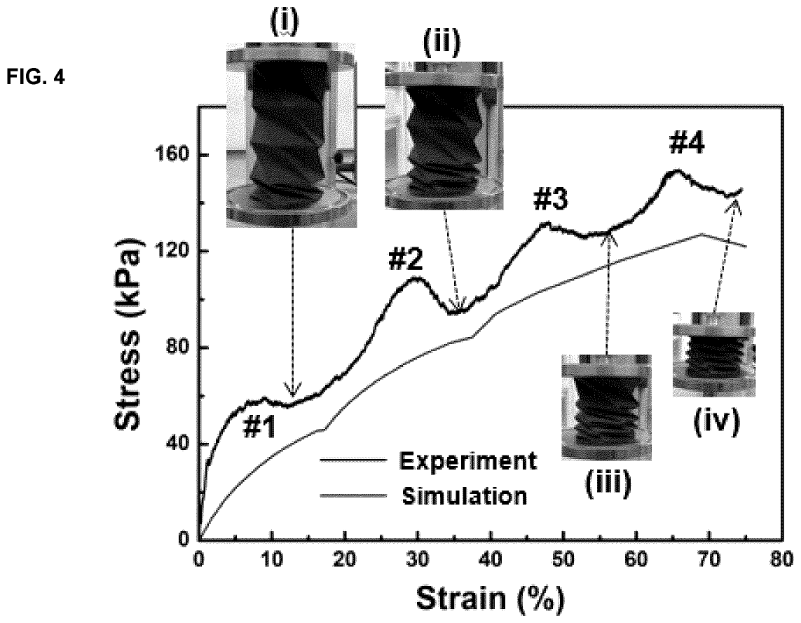

Turning to FIG. 4, there is shown a stress-strain profile exhibited by airbag 10 during a compression cycle. During the compression, the stress increases cyclically (from (i)-(iv)) depending on the applied strain. Each sudden increase in stress generally correlates to a different horizontal row of folding portions 46 undergoing folding. For example, at the first stress sub-peak (#1), it can be seen that this first sub-peak corresponds to the lowermost row of folding portions 46 undergoing folding (image (i)). At the second stress sub-peak (#2), it can be seen that this second sub-peak corresponds to the second lowest row of folding portions 46 undergoing folding (image (ii)). At the third stress sub-peak (#3), it can be seen that this third sub-peak corresponds to the second highest row of folding portions 46 undergoing folding (image (iii)). And, at the fourth stress sub-peak (#4), it can be seen that this fourth sub-peak corresponds to the uppermost row of folding portions 46 undergoing folding (image (iv)).

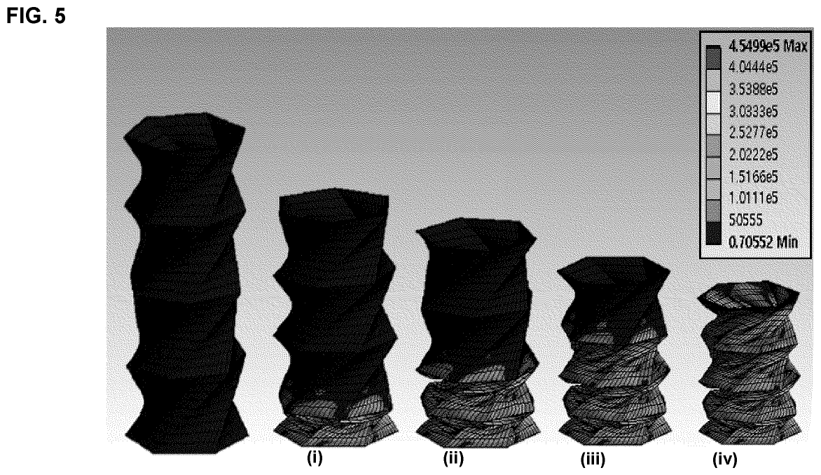

Thus, the airbag 10 exhibits five different stable states at roughly 0%, 15%, 35%, 55%, and 75% of applied strain. As can be seen in FIG. 5, a simulation performed with finite element analysis (FEA) of airbag 10 shows that airbag 10 is generally stressed at its fold lines 41 rather than along polygonal surface portions 43. As a result, when airbag 10 is exposed to cyclic, compressive loading, fatigue accumulated in airbag 10 may be relatively smaller when compared, for example, to a more traditional ventilator airbag. In particular, with more traditional ventilator airbags, the stress-stain curve shows a more linear relationship, indicating that the applied stress is applied to the entire airbag instead of being gradually applied to specific regions of the airbag. As a result, the mechanical reliability of airbag 10 may be improved since the applied stress is concentrated at fold lines 41 of each row of folding portions 46, with folding portions 46 being generally better configured to absorb the applied stress.

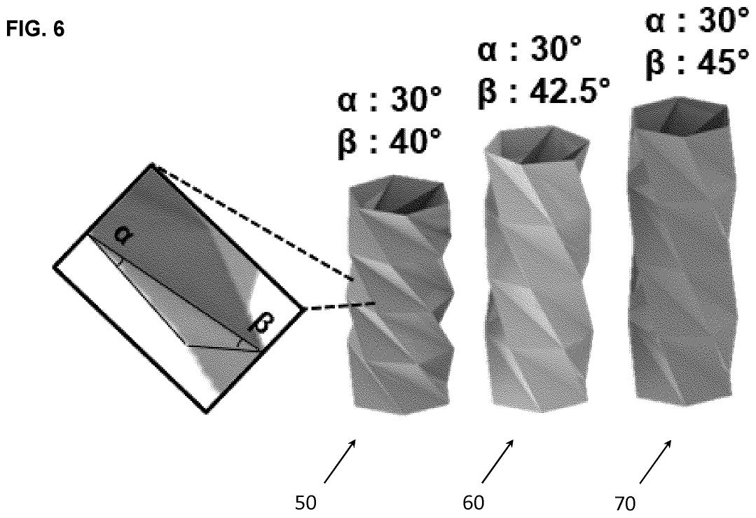

As noted above, angles α and β defined by each triangular surface portion 43 can be adjusted prior to manufacturing of airbag 10 in order to tune parameters of airbag 10. For example, α and β may be adjusted to alter the internal volume defined by airbag 10, or the mechanical rigidity of airbag 10. As can be see in FIG. 6, different airbags 50, 60, and 70 can be arrived at using different values for α and β. Generally, decreasing α and increasing β results in an airbag having a greater internal volume. It was found that setting α to less than 30° generally restricted the ability of the airbag to effectively fold and unfold.

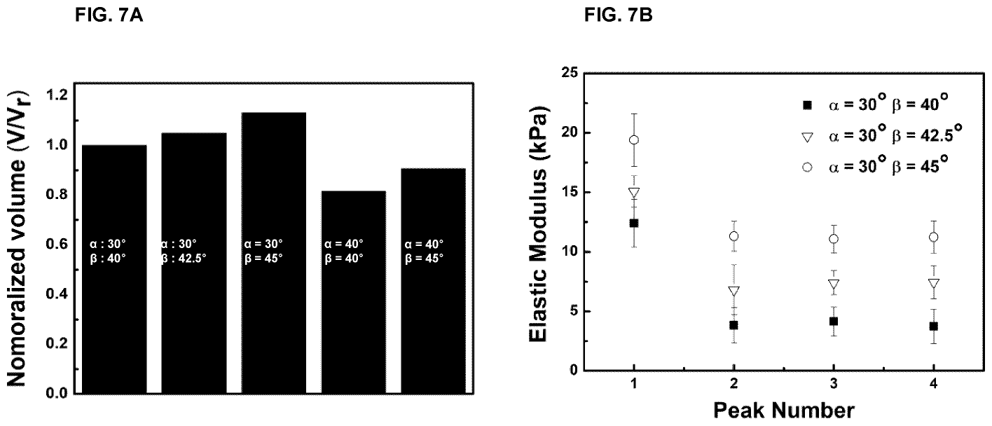

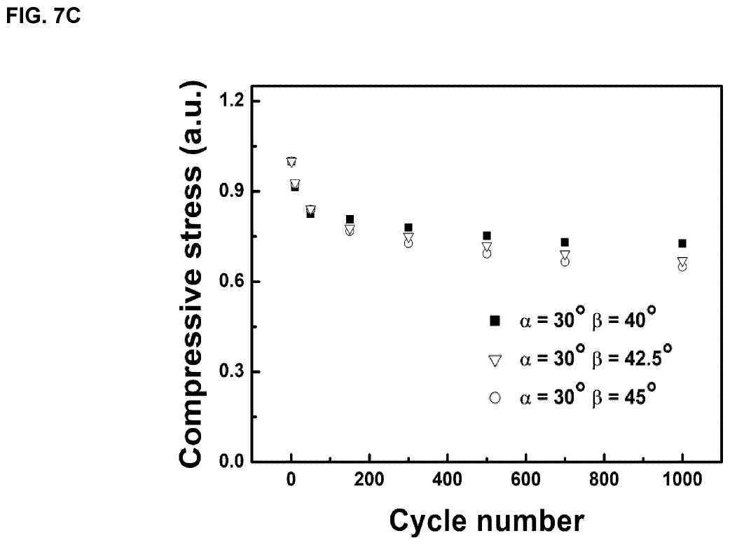

Changes to the normalized volume of the airbag, for different values of α and β, are shown in FIG. 7A. Changes to the elastic modulus of the airbag, for different values of α and β, are shown in FIG. 7B. As can be seen, an airbag with higher α and β angles has a higher elastic modulus indicating greater rigidity. The corresponding results of a cyclic compression test are shown in FIG. 7C. Generally, it may be desired for the airbag to have greater volume for a given mechanical resistance to airflow generation. Therefore, as can be seen from FIGS. 7A-7C, it was found that selecting α=30° and β=42.5° led to an airbag with median values for volume and elastic modulus. According to some embodiments, α may be selected to be between 30° and 34°, and β may be selected to be between 38° and 45°.

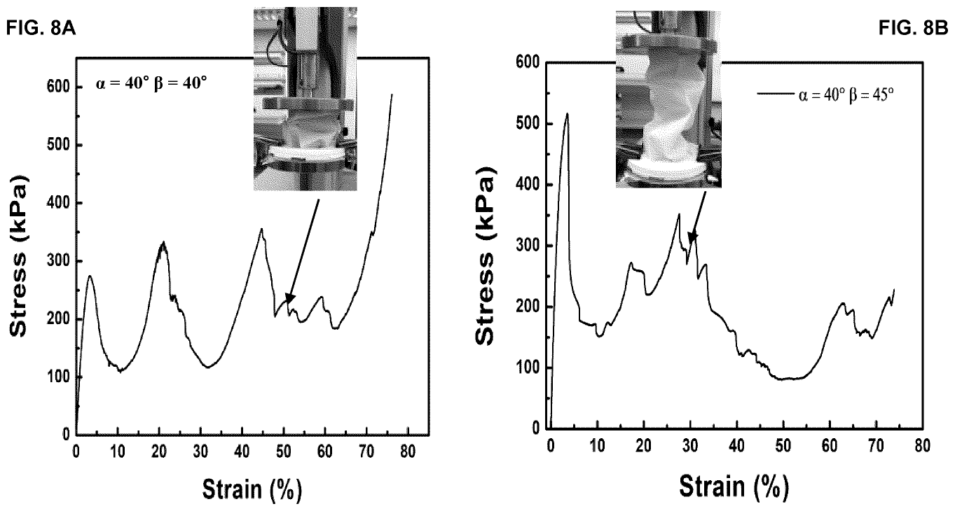

FIGS. 8A and 8B are plots of stress as a function of strain for different airbags with different values for angles α and β, according to embodiments of the disclosure. As can be seen, for α=40° and β=40°, and for α=40° and β=45°, the airbags did not fold properly during compression.

In addition to the angles α and β, other parameters of the airbag may be adjusted in order to further tune the airbag. For example, the diameter of the airbag, the height of the airbag, and/or the number of rows of folding portions may be adjusted.

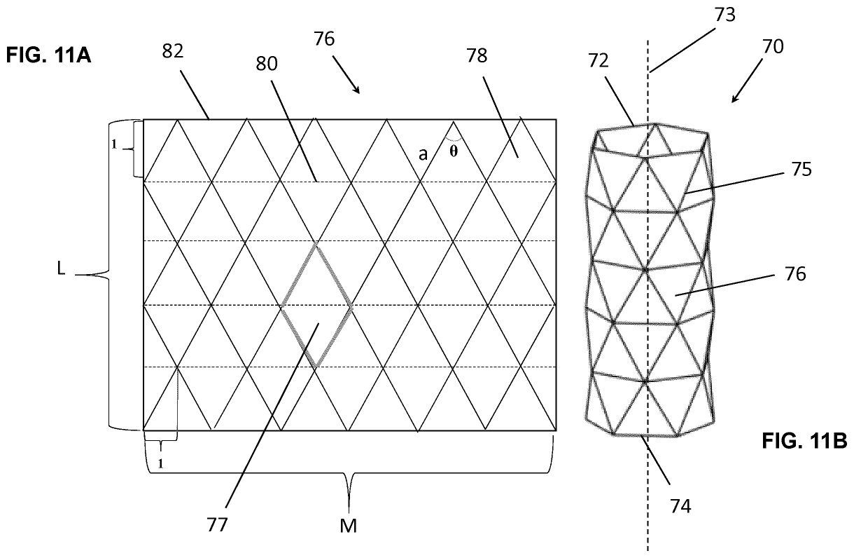

In addition to the Kresling pattern shown in FIG. 3, an airbag may be formed according to other patterns of fold lines. For example, turning to FIGS. 11A and 11B, there is shown an airbag 70 defining a longitudinal axis 73 and having a foldable wall 76 extending between a first end 72 and a second end 74, according to another embodiment of the disclosure. According to this embodiment, the pattern of fold lines 75 on the outer surface of wall 76 may comprise a Yoshimura pattern, shown in more detail in FIG. 11A. FIG. 11A shows in particular the three-dimensional wall 76 of FIG. 11B transposed to a two-dimensional surface. Two-dimensional wall 76 is shaped as a rectangle comprising an arrangement of folding portions 77. Each folding portion 77 is parallelogram-shaped and is defined by four outwardly-folding fold lines 82 and one inwardly-folding fold line 80 extending from a first corner of the parallelogram defined by folding portion 77 to a second, opposite corner of the parallelogram defined by folding portion 77. Furthermore, inwardly-folding fold line 80 extends perpendicularly to longitudinal axis 73 defined by airbag 70.

Each foldable portion 77 defined on two-dimensional wall 76 comprises a pair of planar, polygonal (in this case, triangular) surface portions 78 defined by the intersection of the inwardly folding fold line 80 with the four outwardly folding fold lines 82. The totality of interconnected, triangular surface portions 78 form the outer surface of wall 76. Each triangular surface portion 78 is defined by an angle, θ=600. During compression of airbag 10, first end 72 is not rotated relative to second end 74 about longitudinal axis 73.

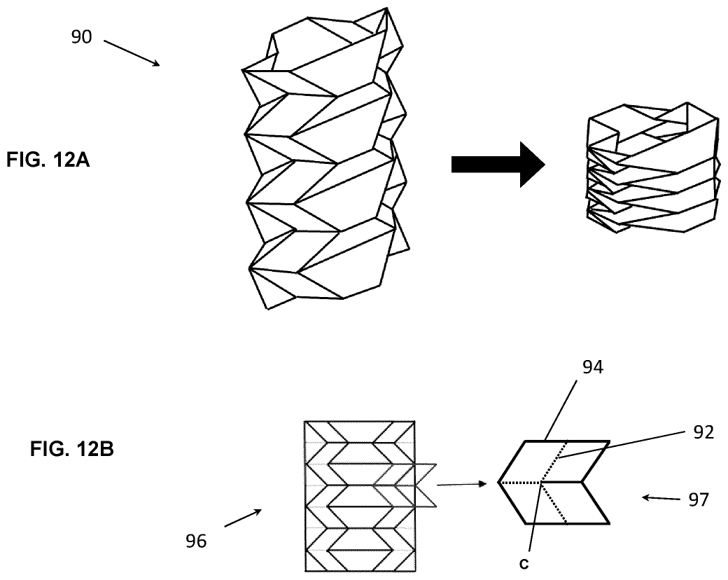

In addition to the Yoshimura pattern shown in FIG. 11A, an airbag may be formed according to still other patterns of foldable portions. For example, turning to FIGS. 12A and 12B, there is shown an airbag 90 for a ventilator, according to an embodiment of the disclosure. Airbag 90, shown in FIG. 12A, has a corresponding foldable wall 96 shown in FIG. 12B. According to this embodiment, the pattern of fold lines on the outer surface of wall 96 comprises a Tachi-Miura pattern. FIG. 12B shows in particular the three-dimensional wall 96 of FIG. 12A transposed to a two-dimensional surface. Two-dimensional wall 96 is shaped as a rectangle comprising an arrangement of folding portions 97. Each folding portion 97 is defined by six outwardly-folding fold lines 94 and three inwardly-folding fold lines 92. The three inwardly-folding fold lines 92 meet at a point O₁ as can be seen in FIG. 12B. During compression of airbag 90, airbag 90 does not rotate about the longitudinal axis defined by airbag 90.

It will be recognized by the skilled person that any number of suitable fold line patterns may be used in order to form an airbag wall according to the present disclosure.

During operation of ventilator 100, control parameters, such as the speed of linear actuation of airbag 10, the volume of air delivered per cycle, and the frequency of each compression cycle, are displayed to users through a display (not shown). Such control parameters may be tuned by a user through appropriate interaction with the controller. For example, the volume of air delivered per cycle may be adjusted by controlling the extent to which airbag 10 is linearly compressed.

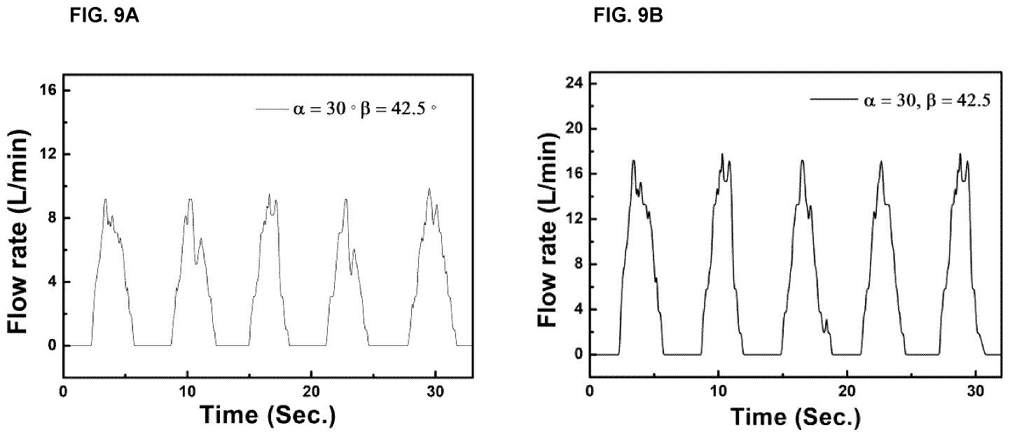

Turning to FIGS. 9A and 9B, there are shown plots of airflow rate as a function of time for an airbag with α=30° and β=42.5°. FIG. 9A shows airflow rate for a 400 mL airbag, and FIG. 9B shows airflow rate for a 600 mL airbag (which may be used for an average adult male patient).

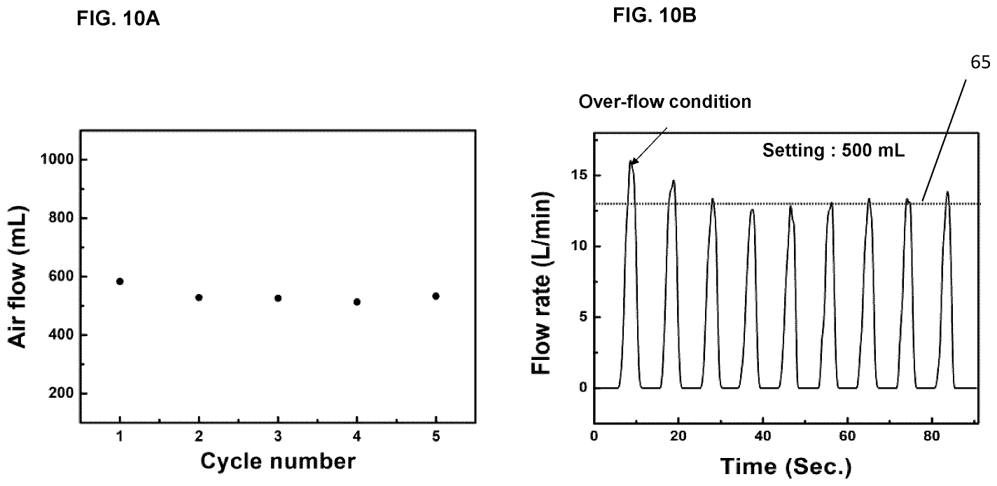

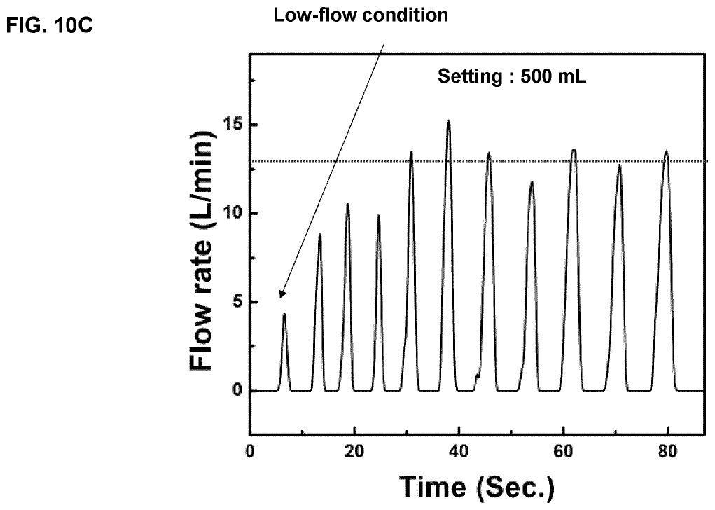

FIG. 10A shows airflow volume as a function of compression cycle, while FIG. 10B shows airflow rate as a function of time. Ventilator 100 may incorporate a safety mechanism whereby, if the airflow rate is determined to be too high, the rate of compression of airbag 10 may be automatically lowered. The volume of airflow may also be automatically tuned based on readings taken by the flow sensor. Further still, the extent to which airbag 10 is linearly compressed during successive compression cycles may be tuned based, again, on readings taken by the flow sensor.

As can be seen in FIG. 10B, an overflow condition (set by a user configurable threshold 65) is met by the first peak in which threshold 65 is exceeded by the measured airflow rate. In response, subsequent compression cycles result in a reduced airflow rate that peaks below threshold 65.

Similarly, if the airflow is determined to be too low, then the rate of compression of airbag 10 may be automatically increased. For example, as can be seen in FIG. 10C, a low-flow condition is met by the first peak in which the measured airflow rate is determined to be significantly lower than threshold 65. In response, subsequent compression cycles result in an increased airflow rate that approaches safety 65.

In order for airbag 10 to be 3D-printed, a variety of different materials may be used. For example, according to embodiments of the disclosure, any one or more of the following various materials may be used: a thermoplastic styrenic block copolymer-based filament; a thermoplastic olefinic elastomer-based filament; a thermoplastic vulcanizate-based filament; a thermoplastic elastomer-based filament; a flexible thermoplastic copolyester-based filament; a thermoplastic polyamide-based filament; a plasticized copolyamide thermoplastic elastomer filament; and a thermoplastic polyurethane-based filament.

In order to print an airbag according as described herein, a design of the foldable wall to be used for the airbag may programmed using, for example, a suitable computer programming tool. The design may be stored on a computer-readable medium and, when read by a 3D printing machine, may enable the 3D printing machine to print the airbag according to the stored design.

The word “a” or “an” when used in conjunction with the term “comprising” or “including” in the claims and/or the specification may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

The terms “coupled”, “coupling” or “connected” as used herein can have several different meanings depending on the context in which these terms are used. For example, as used herein, the terms coupled, coupling, or connected can indicate that two elements or devices are directly connected to one another or connected to one another through one or more intermediate elements or devices via a mechanical element depending on the particular context. The term “and/or” herein when used in association with a list of items means any one or more of the items comprising that list.

As used herein, a reference to “about” or “approximately” a number or to being “substantially” equal to a number means being within +/−10% of that number.

While the disclosure has been described in connection with specific embodiments, it is to be understood that the disclosure is not limited to these embodiments, and that alterations, modifications, and variations of these embodiments may be carried out by the skilled person without departing from the scope of the disclosure.

It is furthermore contemplated that any part of any aspect or embodiment discussed in this specification can be implemented or combined with any part of any other aspect or embodiment discussed in this specification.

1. A ventilator for providing breathable air to a patient, comprising:
an airbag defining a longitudinal axis, comprising a first end, a second end, and a foldable wall extending from the first end to the second end, and being movable from a decompressed state to a compressed state by moving the first end along the longitudinal axis relative to the second end, wherein the wall comprises fold lines formed therein such that, during movement of the airbag from the decompressed state to the compressed state, the wall is folded along the fold lines, and wherein the fold lines define at least one polygonal surface portion of the wall;
an actuator for driving compression and decompression of the airbag; and
a controller for controlling the actuator.

2. The ventilator of claim 1, further comprising a conduit connected to the airbag for delivering the breathable air to the patient.

3. The ventilator of claim 1, wherein the at least one polygonal surface portion comprises interconnected polygonal surface portions of the wall, and wherein the interconnected polygonal surface portions comprise an outer surface of the wall.

4. The ventilator of claim 1, wherein the at least one polygonal surface portion comprises at least one planar polygonal surface portion.

5. The ventilator of claim 1, wherein the fold lines define a Kresling pattern, a Yoshimura pattern, or a Tachi-Miura pattern on an outer surface of the wall.

6. The ventilator of claim 1, wherein the at least one polygonal surface portion comprises at least one triangular surface portion.

7. The ventilator of claim 6, wherein a first angle of the at least one triangular surface portion is from about 30 degrees to about 34 degrees, and wherein a second angle of the at least one triangular surface portion is from about 38 degrees to about 45 degrees.

8. The ventilator of claim 7, wherein the first angle is 30° and the second angle is 42.5°.

9. The ventilator of claim 6, wherein the at least one triangular surface portion does not comprise a right angle.

10. The ventilator of claim 6, wherein the at least one triangular surface portion comprises an obtuse angle.

11. The ventilator of claim 6, wherein the at least one triangular surface portion comprises a right angle.

12. The ventilator of claim 1, wherein the fold lines comprise inwardly-folding fold lines and outwardly-folding fold lines defining interconnected foldable portions of the wall, each inwardly folding fold line being closer to the longitudinal axis than each outwardly folding fold line.

13. The ventilator of claim 12, wherein each foldable portion is defined by four outwardly-folding fold lines and one inwardly folding fold line.

14. The ventilator of claim 13, wherein the four outwardly folding fold lines define a parallelogram, and wherein the inwardly folding fold line extends from a first corner of the parallelogram to an opposite, second corner of the parallelogram.

15. The ventilator of claim 13, wherein the inwardly folding fold line is perpendicular to the longitudinal axis.

16. (canceled)

17. The ventilator of claim 12, wherein each foldable portion is defined by multiple inwardly-folding fold lines meeting at a point.

18. The ventilator of claim 17, wherein each foldable portion is defined by six outwardly-folding fold lines, and wherein the multiple inwardly-folding fold lines meeting at the point consist of three inwardly-folding fold lines.

19. (canceled)

20. (canceled)

21. The ventilator of claim 1, wherein the wall comprises a thermoplastic.

22. (canceled)

23. (canceled)

24. (canceled)

25. (canceled)

26. (canceled)

27. An airbag comprising:
a first end;
a second end; and
a foldable wall extending from the first end to the second end,
wherein the airbag defines a longitudinal axis and is movable from a decompressed state to a compressed state by moving the first end along the longitudinal axis relative to the second end,
wherein the wall comprises fold lines formed therein such that, during movement of the airbag from the decompressed state to the compressed state, the wall is folded along the fold lines, and
wherein the fold lines define at least one polygonal surface portion of the wall.

28. (canceled)

29. (canceled)

30. (canceled)

31. (canceled)

32. (canceled)

33. (canceled)

34. (canceled)

35. A method of making an airbag for a ventilator, comprising printing the airbag using a three-dimensional printer, wherein the printed airbag defines a longitudinal axis, comprises a first end, a second end, and a foldable wall extending from the first end to the second end, and is movable from a decompressed state to a compressed state by moving the first end along the longitudinal axis relative to the second end, wherein the wall comprises fold lines formed therein such that, during movement of the airbag from the decompressed state to the compressed state, the wall is folded along the fold lines, and wherein the fold lines define at least one polygonal surface portion of the wall.

36. (canceled)

37. (canceled)

38. (canceled)

39. (canceled)

40. (canceled)

41. (canceled)

42. (canceled)

43. (canceled)
